(19) United States
(12) Patent Application Publication
(10) Pub. No.: US 2019/0307104 A1
(43) Pub. Date: Oct. 10, 2019

(54) CANINE DIAPER DEVICE

(71) Applicants: Aleisia Dragon, Colorado Springs, CO (US); Chris Richards, Colorado Springs, CO (US)

(72) Inventors: Aleisia Dragon, Colorado Springs, CO (US); Chris Richards, Colorado Springs, CO (US)

(21) Appl. No.: 16/374,844

(22) Filed: Apr. 4, 2019

Related U.S. Application Data
(60) Provisional application No. 62/653,198, filed on Apr. 5, 2018.

(51) Int. Cl. A01K 23/00; A61F 13/49

(52) U.S. Cl. CPC A01K 23/00; A61F 2013/15186; A61F 13/49

(57) ABSTRACT

A canine diaper with re-useable and removable absorption pad may include a resilient body band which may include a resilient fabric material. The body band may have a first side configured for contacting the body of a desired animal, preferably a dog, and a second side which may be positioned opposite from the first side and away from the body of the animal. A liner may be coupled to the first side at a central region on the body band. A pocket panel may be coupled to the liner and/or first side to form a pocket cavity between the liner and pocket panel. An absorption pad may be removably received in the pocket cavity which may be positioned in contact with the urine emitting organ of the animal. A retainer may be removably coupled to the second side via a fastener, and the retainer may be configured to govern access to the pocket cavity.

### CROSS REFERENCE TO RELATED APPLICATIONS

This application claims priority to and the benefit of the filing date of U.S. Provisional Application No. 62/653,198, filed on Apr. 5, 2018, entitled “CANINE DIAPER WITH RE-USEABLE AND REMOVABLE ABSORPTION PAD”, which is hereby incorporated by reference in its entirety.

### FIELD OF THE INVENTION

This patent specification relates to the field of pet diaper devices. More specifically, this patent specification relates to a pet diaper that is configured to capture urine from dogs during marking and urinating behaviors as well as urinary incontinence for senior dogs.

### BACKGROUND

Dog owners are particularly aware that it is common for male dogs to perform marking and urinating behaviors which can be especially troublesome when done indoors. This is a common problem for puppies, adult dogs, and even senior dogs. While there are dog diaper and wearable urine collection devices, they suffer from many drawbacks. Many of these devices include hook and loop fasteners, such as Velcro, which sticks and gets tangled up when laundered with other items, such as clothing, causing damage to the items. Additionally, the hook and/or loop fasteners tend to wear away after a few washes resulting in the need to frequently replace these devices. Other current devices may claim to be re-usable, but once soiled, they can only be re-used a limited number times after washing before they need to be retired from service because of sanitary purposes and scent. Still other current devices have elastic edges which causes rubbing and digging into the skin which may result in lesions and sometimes painful obstructions. Further currently available devices and wraps become inadequate over time once the dog learns it is able to wiggle, rub their bodies on the carpet or other items to leverage the fasteners of the device on the ground to free themselves of the device.

Therefore, a need exists for a novel pet diaper device. There is also a need for a novel re-usable pet diaper device that is configured to capture urine from dogs during marking and urinating behaviors. A further need exists for a novel pet diaper device which remains securely in place on the pet while not causing rubbing and digging into the skin which may result in lesions and sometimes painful obstructions to the pet.

### BRIEF SUMMARY OF THE INVENTION

A canine diaper device with a removable absorption pad is provided. In some embodiments, the device may include a body band having a first side which may be configured to be positioned in contact with the body of an animal, and the body band may have a second side positioned opposite to the first side. A liner may be coupled to both a first end and a second end of the body band. An inner pocket panel may be coupled to both the first end and the second end of the body band, and the inner pocket panel may be aligned with the liner. The inner pocket panel may be continuous with the first side of the body band, and the inner pocket may include an aperture that may be aligned with the liner. An absorption pad may be removably received in a pocket cavity via an access opening of the pocket cavity. A retainer may be configured to govern access to the pocket cavity. The body band may be positioned around the belly of the animal so that the pocket panel is positioned to cover the animal's urinary organ to collect emitted urine which may be absorbed into the absorption pad. When soiled, the diaper may be washed and reused. Additionally, the absorption pad may be easily exchanged with another absorption pad when desired.

In further embodiments, the device may include a body band having a first side which may be configured to be positioned in contact with the body of an animal, and the body band may have a second side positioned opposite to the first side. A liner may be coupled to both a first end and a second end of the body band. An inner pocket panel may be coupled to both the first end and the second end of the body band, and the inner pocket panel may be aligned with the liner. The inner pocket panel may be continuous with the first side of the body band, and the inner pocket may include an aperture that may be aligned with the liner. An absorption pad may be removably received in a pocket cavity. The absorption pad may include an absorbing material and a waterproof material.

### BRIEF DESCRIPTION OF THE DRAWINGS

Some embodiments of the present invention are illustrated as an example and are not limited by the figures of the accompanying drawings, in which like references may indicate similar elements and in which:

FIG. 1—FIG. 1 depicts a top perspective view of an example of a canine diaper device secured to a dog according to various embodiments described herein.

FIG. 2—FIG. 2 illustrates a bottom perspective view of an example of a canine diaper device secured to a dog according to various embodiments described herein.

FIG. 3—FIG. 3 shows a side perspective view of an example of a canine diaper device with absorption pad removed from the pocket cavity according to various embodiments described herein.

FIG. 4—FIG. 4 depicts a side perspective view of an example of a canine diaper device with an absorption pad positioned in the pocket cavity according to various embodiments described herein.

FIG. 5—FIG. 5 illustrates a side perspective, interior view of an example of a canine diaper device according to various embodiments described herein.

FIG. 6—FIG. 6 shows a partial plan view of an example of a canine diaper device showing a pocket panel and aperture with an absorption pad according to various embodiments described herein.

FIG. 7—FIG. 7 depicts a partial sectional, through line 7-7 shown in FIG. 6, elevation view of an example of a canine diaper device according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

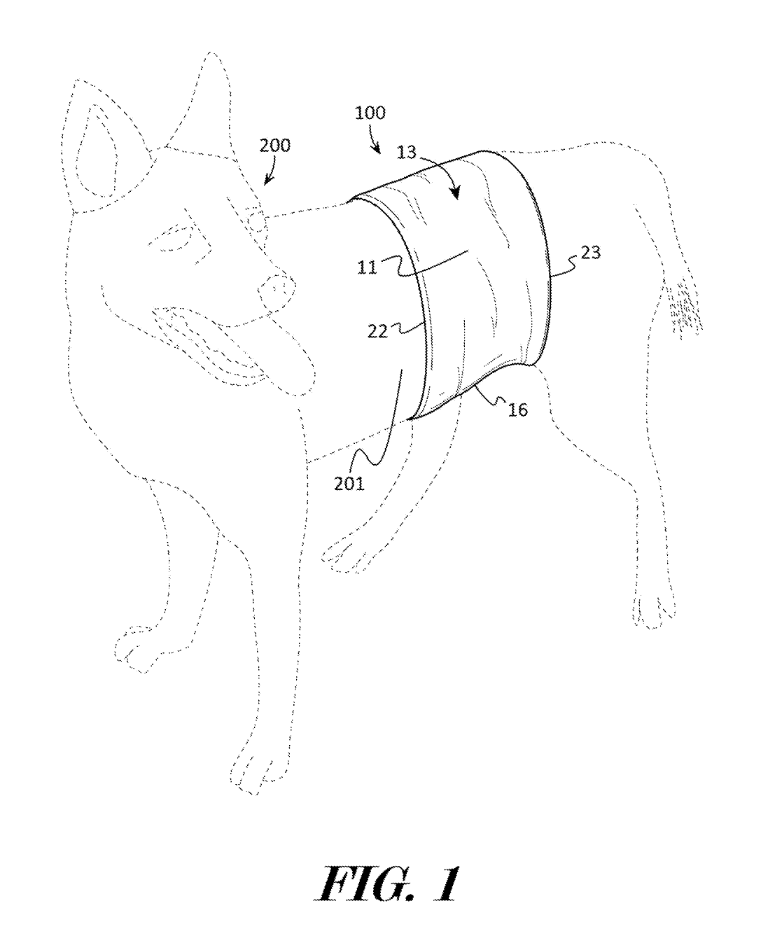

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts of the invention. Hence, specific dimensions and other physical characteristics relating to the embodiments disclosed herein are not to be considered as limiting, unless expressly stated otherwise.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A new pet diaper device is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

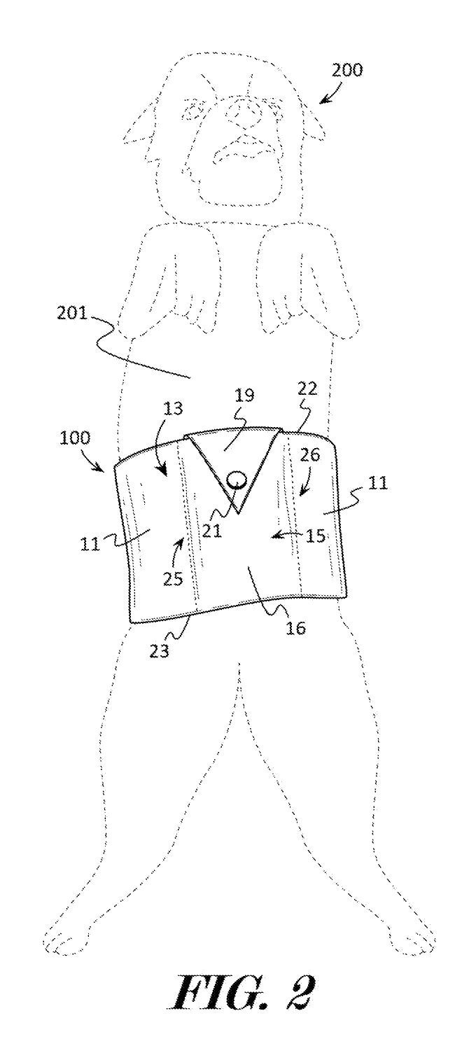

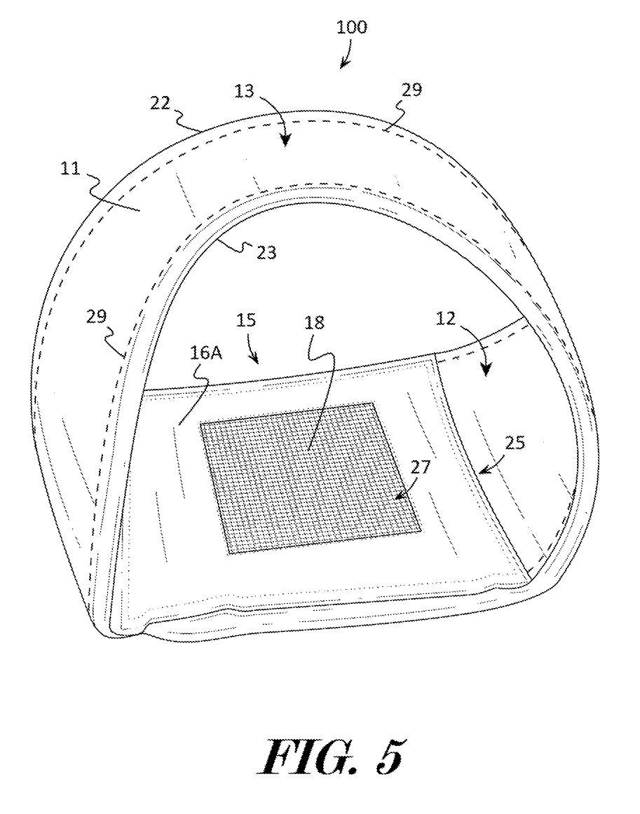

The present invention will now be described by example and through referencing the appended figures representing preferred and alternative embodiments. FIGS. 1-7 illustrate examples of a canine diaper device (“the device”) 100 according to various embodiments. In some embodiments, the device 100 may have a continuous annular shape which may be positioned on an animal 200 so as to encircle portions of the body 201 of the animal 200. The device 100 may include a body band 11 having a first side 12 configured to be positioned in contact with the body 201 of an animal 200, such as a canine, and the body band 11 may have a second side 13 positioned opposite to the first side 12. A liner 14 may be coupled to both a first end 25 and a second end 26 of the body band 11. An inner pocket panel 16A may be coupled to both the first end 25 and the second end 26 of the body band 11, and the inner pocket panel 16A may be aligned with the liner 14. The inner pocket panel 16A may be continuous with the first side 12 of the body band 11, and the inner pocket 16A may comprise an aperture 27 that may be aligned with the liner 14. An absorption pad 18 may be removably received in a pocket cavity 17 via an access opening 28, and a retainer 19 may be configured to govern access to the pocket cavity 17.

In further embodiments, the device 100 may include a resilient body band 11 which may comprise a resilient fabric material. The body band 11 may have a first side 12 which may be positioned in contact with the body 201 of a desired animal 200, preferably a dog, and a second side 13 which may be positioned opposite from the first side 12 and away from the body 201 of the animal 200. A liner 14 may be coupled to the first side 12 at a central region 15. A pocket panel 16 may be coupled to the liner 14 and/or first side 12 to form a pocket cavity 17 between the liner 14 and pocket panel 16. An absorption pad 18 may be removably received in the pocket cavity 17. A retainer 19 may be removably coupled to the second side 13 via a fastener 21, and the retainer 19 may be configured to govern access to the pocket cavity 17. The body band 11 may be positioned around the belly of the animal 200 so that the pocket panel 16 is positioned to cover the animal's 200 urinary organ to collect emitted urine which may be absorbed into the absorption pad 18. When soiled, the device 100 may be washed and reused. Additionally, the absorption pad 18 may be easily exchanged with another absorption pad 18 when desired.

The device 100 may comprise one or more body bands 11 which may be used to position and to secure the device 100 to the animal 200 so that the pocket cavity 17 and an absorption pad 18 contained therein may be positioned on the belly of the animal 200 to cover the animal's urinary organ. In preferred embodiments, the device 100 may comprise a body band 11 having a width defined by a first edge 22 and an opposing second edge 23. In further preferred embodiments, a body band 11 may comprise a width of approximately between two and twelve inches and more preferably, between four and eight inches.

The body band 11 may comprise a first end 25 and a second end 26 which may be coupled to a central region 15 of the device 100. Generally, a pocket cavity 17, liner 14, pocket panel(s) 16, and an absorption pad 18 may be disposed in the central region 15, and the central region 15 may be positioned to cover the urinary organ of an animal 200. In some embodiments, the body band 11 may have a continuous annular shape so that the body band 11 extend through the central region 15 and does not terminate at the first end 25 and second end 26. In other embodiments, a body band 11 may not be continuous and may terminate at the first end 25 and second end 26.

In some embodiments, a body band 11 may be configured to be elastic, resilient, and/or stretchy so as to conform to the anatomy of a desired animal 200 without compromising the health of the animal 200 as opposed to currently available animal wraps have elastic edges causing rubbing and digging into the skin which causes lesions and painful bowel obstructions. In preferred embodiments, a body band 11 may be configured to be resilient so that the portions of the body band 11 between the first end 25 and second end 26 are able to be stretched away from each other. An elastic, resilient, and/or stretchy body band 11 may preferably have an un-stretched length that may be less than the length of the waist/belly of the animal 200 so that when the body band 11 is positioned around the waist/belly of the animal 200, the body band 11 may be tensioned around the animal's 200 waist/belly and be frictionally retained in position.

In preferred embodiments, a resilient body band 11 may be made from or may comprise fabric of natural and/or synthetic materials such as, for example, nylon, satin, spandex, cotton, silk, spandex, and polyester or blends thereof, woven materials such as felt, non-woven materials including non-woven spunbond or carded webs of polypropylene, polyethylene, nylon, polyester, a non-woven web of cellulosic fibers, textile fibers such as rayon fibers, or a blend of cellulosic and textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers, and natural fibers (e.g., wood or cellulose) and the body band 11 may include elastic stitching 29 which may enable the body band 11 to be resilient. Elastic stitching 29 may comprise an elastic sewing thread, typically comprising blends of polyester/rubber and the like, which may be used to couple and gather portions of a resilient body band 11.

In further embodiments, a resilient body band 11 may be made from or may comprise a resilient fabric material comprising spandex or spandex blends, such as cotton-spandex blend, polyester-spandex blend, stretch satin, stretch velvet, nylon spandex fabric, rayon knit spandex, bamboo rayon, stretch denim, and cotton poplin stretch fabric. In still further embodiments, a body band 11 may be made from or may comprise an elastic fabric material such as braided elastic fabric materials, woven elastic fabric materials, and knitted elastic fabric materials. In still further embodiments, a resilient body band 11 may be made from or may comprise an elastic material such as neoprene, cotton swimwear elastic, roll elastic, non-roll elastic, rubber, silicone, elastic plastics, elastic silicone, elastic rubbers, silicone rubbers, or any other suitable elastic, elastomer, or other resilient material.

The device 100 may comprise a liner 14 which may be coupled to the body band 11 at the first end 25 and second end 26 and may extend between the first end 25 and second end 26. Preferably, the liner 14 may be coupled proximate to the second side 13 at the central region 15 on the device 100. In some embodiments, the body band 11 may be configured to be generally annular or cylindrical and continuous in shape and the liner 14 may be coupled to the body band 11 to cover portions of the second side 13. In alternative embodiments, the body band 11 may not be continuous in shape but may have two separated opposing ends 25, 26, which may be coupled to opposing ends of a liner 14 to form a generally cylindrical shape. The liner 14 may be made from a generally waterproof material which may prevent liquids, such as urine, from passing through the liner 14. A waterproof material may be generally flexible and may include nylon, polyurethane, vinyl, polyethylene, polyvinyl chloride, rubber, silicone, or any other suitable generally waterproof and flexible material.

The device 100 may comprise one or more pocket panels 16, such as an inner pocket panel 16A and an outer pocket panel 16B, which may be coupled to the liner 14 and/or first side 12 of the body band 11 to form a pocket cavity 17 between the liner 14 and pocket panel 16. Preferably, a pocket panel 16 may be generally rectangular in shape and three perimeter sides of the pocket panel 16 may be coupled to the liner 14 and/or first side 12 of the body band 11 to form a generally rectangular shaped pocket cavity 17. In other embodiments, a pocket panel 16 and pocket cavity 17 may be configured in an oval shape, triangular shape, circular shape, or any other shape. In preferred embodiments, a pocket panel 16 may be made from or may comprise a recycled cotton fabric material. In further embodiments, a pocket panel 16 may be made from or may comprise synthetic fabrics such as polyester, polyamide, acrylic, nylon, rayon, acetate, spandex, spandex blends, and natural fabrics such as coir, cotton, terry cloth, hemp, jute, linen, ramie, wool, silk, or any other suitable flexible natural or synthetic material including combinations of materials.

In some embodiments, the device 100 may comprise an inner pocket panel 16A which may be positioned and aligned with a liner 14 so as to be relatively proximally positioned to the body 201 of the animal 200 wearing the device 100. An inner pocket panel 16A may be aligned with a liner 14, and the inner pocket panel 16A may be continuous with the first side 25 of the body band 11. In further embodiments, an inner pocket panel 16A and all or portions of the body band 11 may be made of separate materials, and the inner pocket panel 16A may be coupled to both the first end 25 and the second end 26 on the first side 25 the body band 11, such as with stitching, heat bonding, adhesives, or any other coupling method. In other embodiments, an inner pocket panel 16A and all or portions of the body band 11 may be made of a single unit of material so the body band 11 and the inner pocket panel 16A may be integrally formed together. Preferably, an inner pocket panel 16A may be positioned on one or more sides or in contact with the animal's 200 urinary organ to collect emitted urine which may be absorbed through an aperture 27 in the pocket panel 16 and into the absorption pad 18. Generally, an aperture 27 may comprise an opening in the inner pocket panel 16A of any size and shape, such as a rectangular shape, circular shape, or any other shape, which may be positioned over an animal's 200 urinary organ so that emitted urine may be directly absorbed into an absorption pad 18.

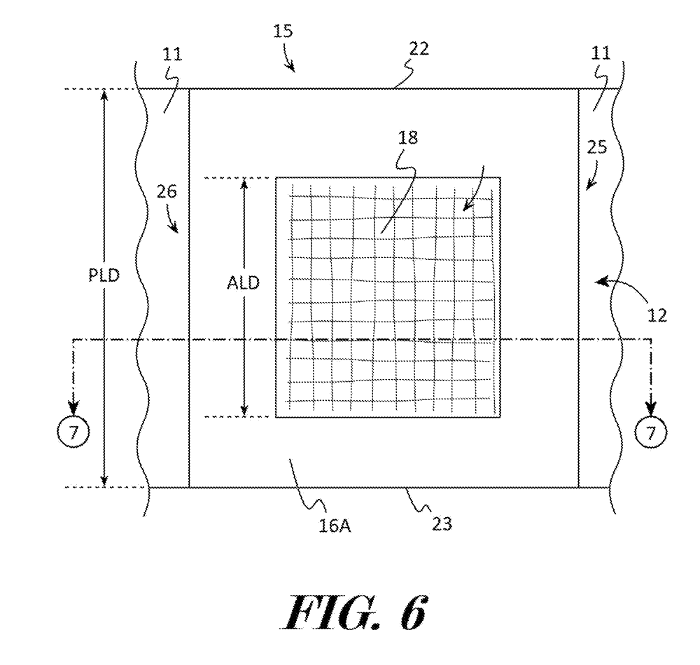

In some embodiments, an inner pocket panel 16A may comprise a panel length dimension (PLD) which may generally describe the distance from opposing ends (such as those coupled to the edges 22, 23, of the body band 11) as shown in FIG. 6. Likewise, an aperture 27 of the inner pocket panel 16A may comprise an aperture length dimension (ALD) which may generally describe a distance parallel to the PLD also oriented from opposing ends of the inner pocket panel 16A as shown in FIG. 6. In preferred embodiments, the ALD may be between 50 to 90 percent of PLD. In other embodiments, an aperture 16 may be configured in any size and shape relative to an inner pocket panel 16A.

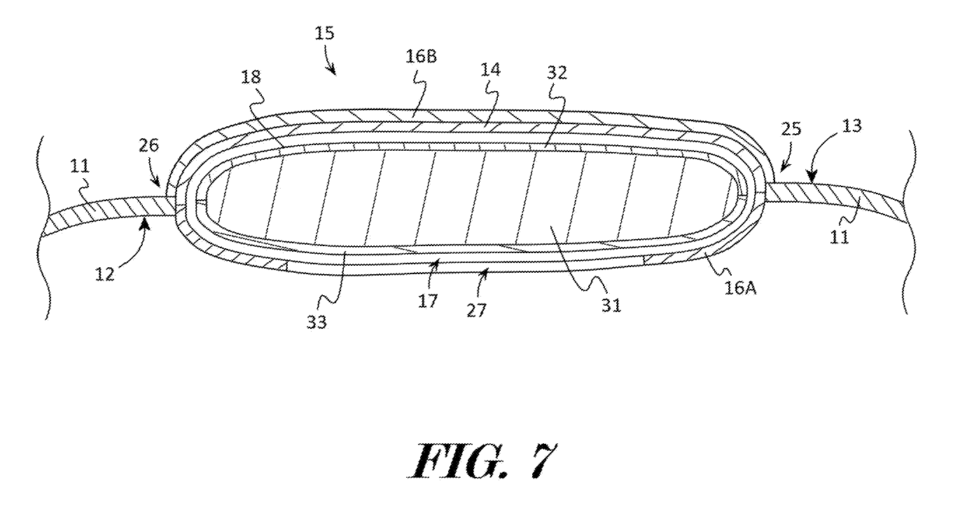

In some embodiments, the device 100 may comprise an outer pocket panel 16B which may be positioned and aligned with a liner 14 so as to be relatively distally positioned to the body 201 of the animal 200 wearing the device 100. Generally, an inner pocket panel 16A and an outer pocket panel 16B may be positioned on opposing sides of a pocket cavity 17. An outer pocket panel 16B may be aligned with a liner 14, inner pocket panel 16A, aperture 27, and pocket cavity 16 (as shown in FIG. 7), and the outer pocket panel 16B may be continuous with the second side 26 of the body band 11. In further embodiments, an outer pocket panel 16B and all or portions of the body band 11 may be made of separate materials, and the outer pocket panel 16B may be coupled to both the first end 25 and the second end 26 on the second side 26 the body band 11, such as with stitching, heat bonding, adhesives, or any other coupling method. In other embodiments, an outer pocket panel 16B and all or portions of the body band 11 may be made of a single unit of material so the body band 11 and the outer pocket panel 16B may be integrally formed together. In still further embodiments, the body band 11 may comprise at least two layers of material, with one layer forming the inner pocket panel 16A and another layer forming the outer pocket panel 16B.

The device 100 may comprise one or more absorption pads 18 which may be removably received in the pocket cavity 17. By being removably received in the pocket cavity 17, absorption pad(s) 18 may be easily exchanged with other absorption pad(s) 18 when desired. A pocket cavity 17 may be configured in any size and shape, and preferably, a pocket cavity 17 may be configured with a size and shape that is generally complementary in size and shape to an absorption pad 18 so that absorption pad 18 movement within the pocket cavity 17 may be limited. An absorption pad 18 may be inserted into and removed from the pocket cavity 17 via an access opening 28. An access opening 18 may positioned anywhere on the device 100 such as proximate to ab edge 22, 23.

An absorption pad 18 may be made from or may comprise any material which may be absorbent to liquids such as urine. In preferred embodiments, an absorption pad 18 may comprise an absorbing material 31 that may be sandwiched between a waterproof material 32 and a wicking material 33. In some embodiments, an absorbing material 31 may comprise a material that may be made out of tangled cellulose fibers from bamboo, cotton, viscose and/or poly micro fiber, such as Zorb®. In further embodiments, an absorbing material 31 may be made from or may comprise any other material capable of absorbing urine and other bodily fluids. A waterproof material 32 may comprise any waterproof material, such as which may be used for a liner 14. A wicking material 33 may comprise any material which may be suitable for wicking moisture. Preferably, a wicking material 33 may comprise a one-way wicking material which may substantial be capable of wicking moisture in one direction. For example, one-way wicking material 33 may comprise polyester, or other fabric, in which the inner side of the fabric is treated to be more hydrophobic than the outer side and there is therefore a hydrophilicity gradient through the thickness of the fabric (gradation of surface tension). This causes a capillary pull which moves moisture from the high-hydrophilicity side to the low-hydrophilicity side.

In further embodiments, an absorption pad 18 may be made from or may comprise a washable material such as a recycled cotton fabric material. In still further embodiments, an absorption pad 18 may be made from or may comprise a washable material such as synthetic fabrics such as polyester, polyamide, acrylic, nylon, rayon, acetate, spandex, spandex blends, and natural fabrics such as coir, cotton, terry cloth, hemp, jute, linen, ramie, wool, silk, or any other suitable flexible natural or synthetic material including combinations of materials. In alternative embodiments, an absorption pad 18 may be configured to be disposable and made from or may comprise a non-washable or single use material such as bleached rayon (cellulose made from wood pulp), cotton, plastics, or other generally disposable absorbent material.

In some embodiments, an absorption pad 18 may comprise a tab 34 which may be grasped or otherwise manipulated by a user to facilitate the removal and insertion of the absorption pad 18 out of and into the pocket cavity 17. A tab 34 may be configured in any size and shape, such as a triangular shape, rectangular shape, circular shape, or combinations of shapes. In preferred embodiments, a tab 34 may be made from or may comprise a waterproof material 32. In alternative embodiments, a tab 34 may be configured as a pull ring, pull string, or any other configuration which may be grasped or otherwise manipulated by a user to facilitate the removal and insertion of the absorption pad 18 out of and into the pocket cavity 17.

In preferred embodiments, the device 100 may comprise one or more retainers 19 which may be configured to secure an absorption pad 18 within a pocket cavity 17 or otherwise govern the ability of an absorption pad 18 to be inserted or removed from the pocket cavity 17. In some embodiments, a retainer 19 may comprise a flap or extension of material, preferably a waterproof material, which may be coupled to or integrally formed with the pocket panel 16 or other element of the device 100. In the example of FIGS. 1-4, a retainer 19 may comprise a generally triangular flap of material integral with or otherwise coupled to the pocket panel 16 which may be folded over the entrance to the pocket cavity 17 and removably secured to the body band 11 via a fastener 21 to govern the ability of an absorption pad 18 to be inserted or removed from the pocket cavity 17. In other embodiments, a retainer 19 may comprise a generally rectangular flap, elongated strip, or any other size and shape which may be folded over the access opening 28 of the pocket cavity 17 and removably secured to the body band 11 via a fastener 21 to govern the ability of an absorption pad 18 to be inserted or removed from the pocket cavity 17 via the access opening 28. In alternative embodiments, a retainer 19 may comprise a hook and loop type fastener, a magnetic fastener, a snap fit fastener, or any other suitable connection method which may be configured to secure portions of the pocket panel 16 to the body band 11 and/or liner 14 which are proximate to or which may form the entrance to the pocket cavity 17 to govern the ability of an absorption pad 18 to be inserted or removed from the pocket cavity 17.

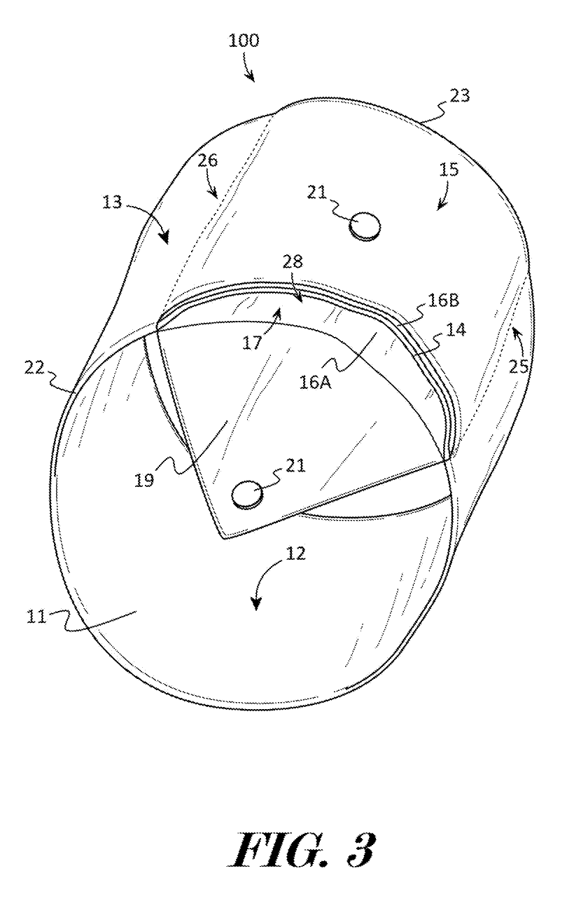

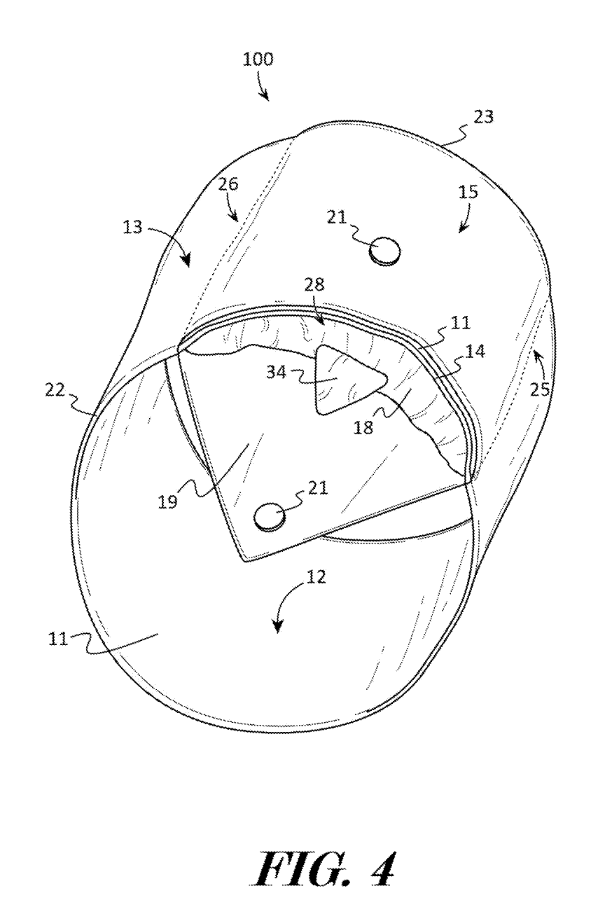

In some embodiments, the device 100 may comprise a retainer 19 which may be removably coupled to the second side 13 via a fastener 21, and the retainer 19 may be configured to govern the ability of a retainer 19 grant or deny access an absorption pad 18 received in the pocket cavity 17. In preferred embodiments, a fastener 21 may comprise a snap fastener which may positioned to allow the retainer 19 to be removably coupled to the second side 13 of the body band 11, liner 14, outer pocket panel 16B, or other element of the device 100 as perhaps best shown in FIGS. 3 and 4. In other embodiments, a fastener 21 may comprise a hook and loop type fastener, zipper, button type fastener, buckle, safety pin, or any other removable type of fastener which may be configured to removably couple a retainer 19 to the body band 11 to govern the ability of a retainer 19 grant or deny access an absorption pad 18 received in the pocket cavity 17.

The body band 11 may be positioned around the belly of the animal 200 so that the absorption pad 18 contained in the pocket cavity 17 is positioned to cover the animal's urinary organ. If and when the animal emits urine, the absorption pad 18 may collect the emitted urine which may be absorbed, optionally through the pocket panel 16, into the absorption pad 18. When soiled, the device 100 and/or absorption pad 18 may be washed and reused. Additionally, the absorption pad 18 may be easily exchanged with another absorption pad 18 when desired. In this manner, the device 100 provides an eco-friendly, non-slip, re-usable animal urine collection wrap with replaceable absorption pads that captures urine from male dogs when marking and peeing inside the house. This is a common problem for puppies, adult dogs, and even senior dogs

While some exemplary shapes and sizes have been provided for elements of the device 100, it should be understood to one of ordinary skill in the art that a body band 11, liner 14, pocket panel 16, pocket cavity 17, absorption pad 18, retainer 19, fastener 21, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiber glass, resins, polymers or any other suitable materials including combinations of materials. Additionally, one or more elements may be made from or comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A canine diaper device having a continuous annular shape, the device comprising:
a body band, the body band having a first side configured to be positioned in contact with the body of an animal, and the body band having a second side positioned opposite to the first side;
a liner coupled to both a first end and a second end of the body band;
an inner pocket panel coupled to both the first end and the second end of the body band, the inner pocket panel aligned with the liner, the inner pocket panel continuous with the first side of the body band, and the inner pocket comprising an aperture that is aligned with the liner;
a pocket cavity having an access opening;
a retainer configured to govern access to the pocket cavity; and
an absorption pad removably received in the pocket cavity.

2. The device of claim 1, wherein the body band and the inner pocket panel are integrally formed together.

3. The device of claim 1, further comprising an outer pocket panel coupled to both the first end and the second end of the body band, the outer pocket panel aligned with the liner, inner pocket panel and the aperture, the outer pocket panel continuous with the second side of the body band.

4. The device of claim 3, wherein the body band and the outer pocket panel are integrally formed together.

5. The device of claim 1, wherein the retainer comprises a snap fastener.

6. The device of claim 1, wherein the absorption pad comprises an absorbing material that is sandwiched between a waterproof material and a wicking material.

7. The device of claim 7, wherein the wicking material is a one-way wicking material.

8. The device of claim 1, wherein the inner pocket panel comprises a panel length dimension (PLD) and the aperture comprises an aperture length dimension (ALD), and wherein ALD is between 50 to 90 percent of PLD.

9. The device of claim 1, wherein the body band is configured to be resilient so that portions of the body band between the first end and second end are able to be stretched away from each other.

10. The device of claim 9, wherein the body band comprises a resilient fabric material.

11. The device of claim 9, wherein the body band comprises elastic stitching.

12. A canine diaper device having a continuous annular shape, the device comprising:
a body band, the body band having a first side configured to be positioned in contact with the body of an animal, and the body band having a second side positioned opposite to the first side;
a liner coupled to both a first end and a second end of the body band;
an inner pocket panel coupled to both the first end and the second end of the body band, the inner pocket panel aligned with the liner, the inner pocket panel continuous with the first side of the body band, and the inner pocket comprising an aperture that is aligned with the liner;
a pocket cavity; and
an absorption pad removably received in the pocket cavity, the absorption pad comprising an absorbing material and a waterproof material.

13. The device of claim 12, wherein the absorbing material is sandwiched between the waterproof material and a one-way wicking material.

14. The device of claim 12, further comprising an outer pocket panel coupled to both the first end and the second end of the body band, the outer pocket panel aligned with the liner, inner pocket panel and the aperture, the outer pocket panel continuous with the second side of the body band.

15. The device of claim 12, further comprising a retainer configured to govern access to the pocket cavity

16. The device of claim 15, wherein the retainer comprises a snap fastener.

17. The device of claim 12, wherein the inner pocket panel comprises a panel length dimension (PLD) and the aperture comprises an aperture length dimension (ALD), and wherein ALD is between 50 to 90 percent of PLD.

18. The device of claim 12, wherein the body band is configured to be resilient so that the first end and second end are able to be stretched away from each other.

19. The device of claim 18, wherein the body band comprises a resilient fabric material.

20. The device of claim 18, wherein the body band comprises elastic stitching.
